(19) United States
(12) Patent Application Publication
(10) Pub. No.: US 2019/0298190 A1
(43) Pub. Date: Oct. 3, 2019

(54) PULSE DETECTION, MEASUREMENT AND ANALYSIS BASED HEALTH MANAGEMENT SYSTEM, METHOD AND APPARATUS

(71) Applicants: Sushanth POOJARY, Mumbai (IN); Indian Institute of Technology Bombay, Maharashtra (IN)

(72) Inventors: Sushanth POOJARY, Mumbai (IN); Pradip GADKINE, Nagpur (IN); Santosh NORONHA, Mumbai (IN)

(21) Appl. No.: 16/346,764

(22) PCT Filed: Dec. 11, 2017
(86) PCT No.: PCT/IN17/50587

§ 371 Date: May 1, 2019

(30) Foreign Application Priority Data
Dec. 9, 2016 (IN) 201621037349

(51) Int. Cl. A61B 5/021; A61B 5/02; A61B 5/022; A61B 5/024; A61B 5/00

(52) U.S. Cl. CPC A61B 5/02108; A61B 5/02007; A61B 5/022; A61B 5/02444; A61B 5/02427; A61B 5/7203; A61B 5/02405

(57) ABSTRACT

A system, apparatus and method for managing health of different individuals by using pulse detection and analysis is described. In one embodiment herein, various pulse qualitative measurements such as Pulse Rate Stiffness Index, Augumentation Index, Ejection Duration, Dichrotic Wave Amplitude, Pulse Height Variance, Pulse Width Variance, Relative Crest Time, Dichrotic Wave Time, Time Delay, Pulse Rate Variance and multiple others as described are used. Further, many derivative parameters are derived from these basic parameters. Central aortic pressure is one such parameter. Various such parameters are recorded along with physical parameters of the subject and used to evaluate the health thereof.

### BACKGROUND

### Technical Field

The present invention relates generally to pulse detection, analysis and application thereof to health monitoring and management.

### Background of the Invention

The importance of health management in today's society is well understood. Health management however relies largely on the measurement and oftentimes prediction of parameters that indicate the wellbeing or a likely/ongoing illness. The factors such as accuracy, lead time bias, length time bias, cost and handling requirements also play an important role when it comes to the implementation of a health management system.

Pulse measurement and analysis has been used for health monitoring. For example, Arterial pulse analysis is useful to evaluate the factors responsible for a heart disease even before the onset of a heart condition. However, the cost, accuracy, handling requirements, or skill requirement for interpreting results of available solutions make it difficult to scale. Early detection in many cases, depends on regular monitoring for increased data points, and thus require frequent data collection by the user. The handling requirements in existing solutions put a restriction on frequent usage of the solution and hence affects the efficacy or usability of such solutions. Further, brachial blood pressure is not always a good indicator of the effect of blood pressure-lowering drugs on arterial hemodynamics. Conventionally, the most reliable measurement of central aortic BP is measured using invasive catheters. Non-invasive techniques exist but do not provide accurate results for varying demographics.

Therefore, there is a need of a system, device and/or a method that has increased accuracy and can be scaled to urban as well as rural areas to allow the user or a health administrator with minimum training to use. Such a solution would greatly benefit the public at large.

### SUMMARY OF THE INVENTION

In general, a system, apparatus and method for health management using pulse detection, measurement and analysis are provided. In one embodiment, a cardiovascular health monitoring device is provided comprising atleast one radial artery pressure sensor to receive radial artery pressure waveform, atleast one brachial artery pressure sensor to receive brachial artery pressure waveform and a signal processing module configured to generate at least one of the cardiovascular parameter including peripheral augmentation index, pulse rate variability, arterial stiffness index, ejection duration, central aortic waveform, central aortic blood pressure and central augmentation index for analysis of cardiovascular health, based upon the received brachial artery pressure waveform and the received radial artery pressure waveform.

An aspect of the subject matter is to provide a cardiovascular health monitoring device, wherein the signal processing module generates the ratio of low frequency to high frequency components in the fourier transform of the pulse rate variability, pulse wave velocity among other parameters and use it for cardiovascular health detection, measurement and management.

Another aspect includes receiving physiological parameters of a subject and generating central aortic pressure waveform using the received brachial artery pressure waveform, the received radial artery pressure waveform, the pulse wave velocity and the received physiological parameters of the subject. The cardiovascular health monitoring device as in claim 4, wherein the physiological parameters of the subject includes, age, gender, height and weight.

Another aspect of the invention deals with the construction of the sensor and sensor housing, wherein, the atleast one radial artery pressure sensor and the atleast one brachial artery pressure sensor each comprise of a bottom surface in contact with the skin such that the area of contact with the skin is atleast multiple times that of the width of artery being sensed a sensor housing having cross-sectional area that reduces with height, starting from the bottom surface, wherein the height of the sensor housing is less than the width of the bottom surface of the sensor.

### BRIEF DESCRIPTION OF THE DRAWINGS

Reference will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 is a block diagram illustrating a pulse detector and analyser based health management system according to an embodiment herein.

FIG. 2a is an image illustrating the strap based design as per an embodiment herein.

FIG. 2b is a schematic representation of sensor housing as per an embodiment herein.

FIG. 3 is an image illustrating a pen based design as per an embodiment herein.

FIG. 4 is an image illustrating the handheld design as per an embodiment herein.

FIG. 5 shows a method for health management using pulse detection, measurement and analysis as per an embodiment herein

FIG. 6 shows a method for parameter extraction as per an embodiment herein.

### DETAILED DESCRIPTION OF THE PREFERRED EMBODIMENTS

A system, apparatus and method for health management using pulse detection, measurement and analysis are provided. In one embodiment herein, various pulse qualitative measurements such as Pulse Rate, Stiffness Index, Augumentation Index, Ejection Duration, Dichrotic Wave Amplitude, Pulse Height Variance, Pulse Width Variance, Relative Crest Time, Dichrotic Wave Time, Time Delay, Pulse Rate Variance and multiple others as described are used. Further, many derivative parameters are derived from these basic parameters. Central aortic pressure is one such parameter. Various such parameters are recorded along with physical parameters of the subject and used to evaluate the health thereof.

The pulse measurements are taken using one or more pulse detection and measurement device. One or more of Pulse Rate (bpm), Stiffness Index, Augumentation Index (%), Ejection Duration (ms), Dichrotic Wave Amplitude, Pulse Height Variance, Pulse Width Variance, Relative Crest Time (sec), Dichrotic Wave Time (sec), Time Delay (sec), Pulse Rate Variance of a subject are conditioned then transmitted for processing. The processed data is recorded into subject's data file that contains historical information about the subject's measurements. The processed data is also available for visual inspection on a graphical user interface (GUI) on a real time basis. The recorded data is evaluated for health implications. The result of evaluation may be provided on a communication device such as for example, a personal computer, digital assistant, a mobile phone, a tablet or may be available on the cloud accessible via a browser or an application on a computing device. In another embodiment, the processing as well as the resulting data may be available on a cloud based server.

In another embodiment central aortic pressure waveform of the subject is estimated using the radial pressure waveform, brachial pressure readings and certain characteristics of the subject such as for example, Age, gender, height. Sometimes weight and/or ethnicity may also be used. Based on the above information a transfer function is calculated and central aortic pressure waveform is predicted.

The following description is set forth for the purpose of explanation in order to provide an understanding of the invention. However, it is apparent that one skilled in the art will recognize that embodiments of the present invention, some of which are described below, may be incorporated into a number of different computing systems and devices. Also, various parameters mentioned herein are exemplary and various others obvious to person skilled in the art may be added. The embodiments of the present invention may be present in hardware, software or firmware. The conditioning, processing or evaluation of data may be present in hardware or software made available on a non-transitory computer medium. Structures shown below in the diagram are illustrative of exemplary embodiments of the invention and are meant to avoid obscuring the invention. Furthermore, connections between components within the figures are not intended to be limited to direct connections. Rather, data between these components may be modified, re-formatted or otherwise changed by intermediary components.

Reference in the specification to “one embodiment”, “in one embodiment” or “an embodiment” etc. means that a particular feature, structure, characteristic, or function described in connection with the embodiment is included in at least one embodiment of the invention. The appearances of the phrase “in one embodiment” in various places in the specification are not necessarily all referring to the same embodiment.

### A. Overview

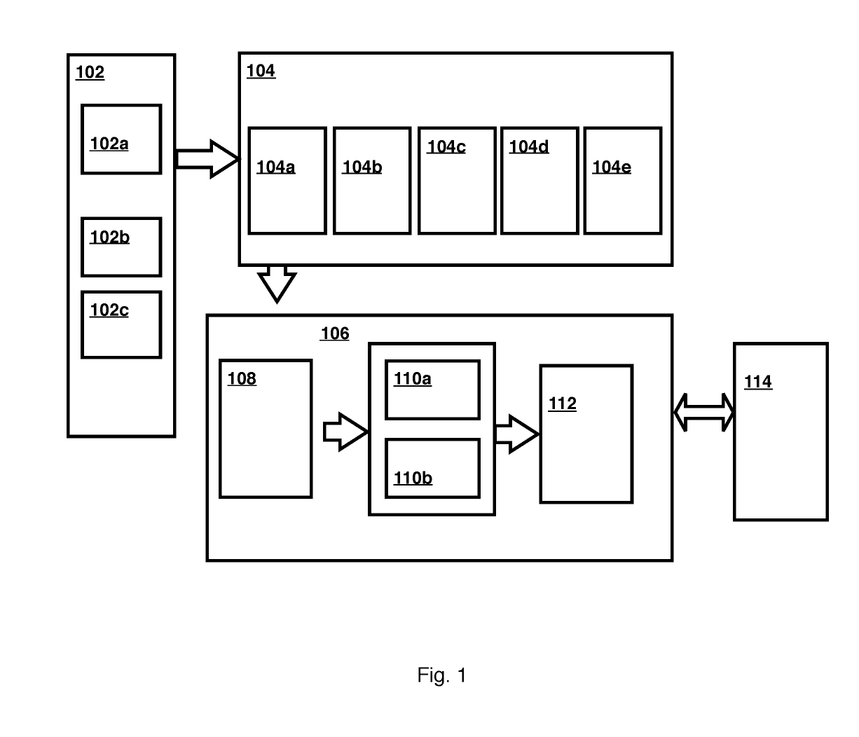

FIG. 1 shows a block diagram illustrating a pulse detector and analyser based health management system 100 according to an embodiment herein. Said system 100 comprises a sensor system 102, a signal conditioning and data acquisition system 104, an evaluation and output system 106 and a remote server 114.

For obtaining the analog signal from a subject, a plurality of pulse detectors such as for example, piezoelectric sensor 102a, LED-diode pair 102b, Brachial artery probe 102c are used. The piezoelectric sensors are used for measuring pressure pulse and an LED-diode pair for volume pulse measurement. In another embodiment, flexible organic thin film pressure sensor may also be used for pressure pulse measurement. The Signal conditioning and data acquisition system 104 is preferably an electronic-equipment having a microcontroller 104d to process the data received from various sensing elements. A low pass filter 104a might be used for cancelling noise. An amplification circuitry 104b amplifies analog signal for better measurement of the waveform. An analog to digital converter 104c digitizes the analog signal for processing in micro-controller 104d. A conditioned and transmission-ready signal is then made available at transmission and relaying unit 104e. In one embodiment, this may be achieved using a Bluetooth transmitter. Other transmission mechanisms such as WiFi or wired systems may also be used.

One or more evaluation and output system 106 may be communicatively coupled to the signal conditioning and data acquisition system 104 for receiving the digital waveform. A signal processing unit 108 is configured to process the digital waveform based on an algorithm. The algorithm detects various features of the digital waveform and produces parameters of the subject. The user gets to see the waveform on a GUI 110a so that she can decide if the waveform collected satisfies the threshold of accuracy for measurement and analysis. The signal may be recorded in the signal recorder 110b. This signal can also be made available in the output unit 112. The output may be in form of a print, audio, visual or audiovisual.

In one embodiment parameters such as for example, Pulse Rate (bpm), Stiffness Index, Augumentation Index (%), Ejection Duration (ms), Dichrotic Wave Amplitude, Pulse Height Variance, Pulse Width Variance, Relative Crest Time (sec), Dichrotic Wave Time (sec), Time Delay (sec), Pulse Rate Variance, type of arterial pulse and multiple others are measured. Further, many derivative parameters are derived from these basic parameters.

A remote server 114 may be configured to receive the parameters and/or health implications on a web-based application, which may be accessible to an end user through his/her communication device. This data can be synced with an electronic medical record (EMR) system using cloud based application on a user computing device. In one embodiment the analysis of the parameters may be done at the remote server 114. In another embodiment the analysis is done locally at the evaluation and output system 106. When done locally, the parameters may be analyzed by a local practitioner or an assistant to provide useful implications. Also, the analysis of the health condition may be updated in real time as more data about the subject is collected by the system. It is to be noted that the health management system described above may be run periodically or on demand. Cardiovascular Health Index of the subject may be measured to give a general metric of cardiovascular health of a person by evaluating the degree of cardiovascular risk factors using peripheral and central blood pressure, pulse wave velocity, peripheral and central augmentation index, arterial stiffness index and other parameters as well as subject's age, gender, height, weight. This will be an overall indicator for subject's cardiovascular health.

### B. Construction

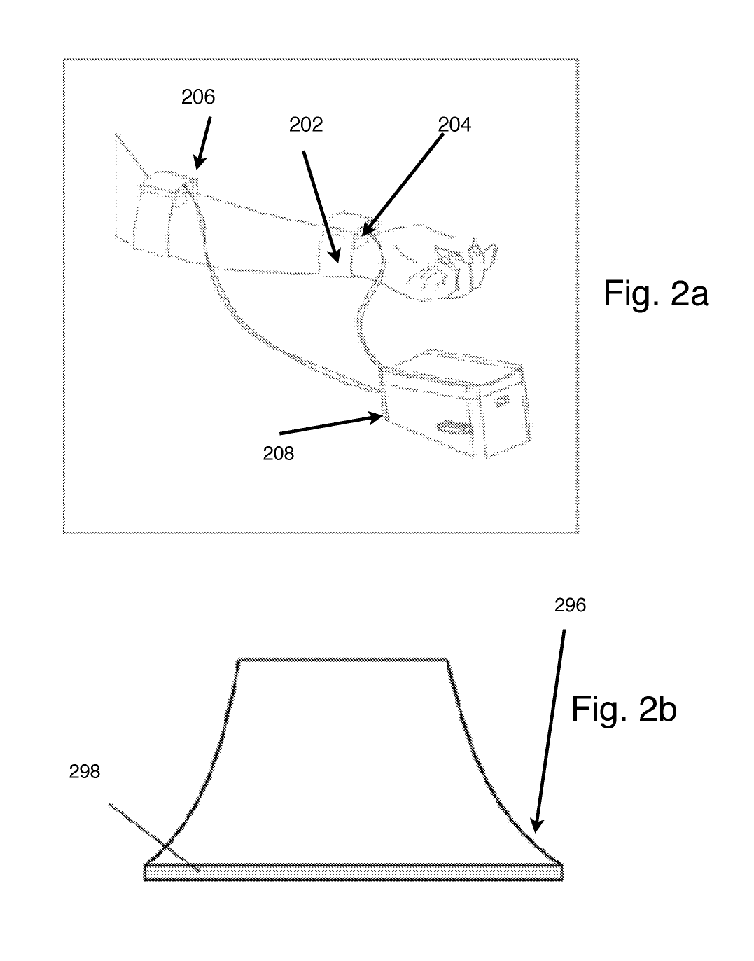

The Sensor system 102 may be made available in a strap based design, a pen-shaped design or a handheld design. FIG. 2a shows an embodiment of strap based design as per one embodiment herein. A strap 202 is provided linked to the sensor system 204. This may be done using a velcro arrangement. In case of the use of a brachial artery probe a separate arrangement 206 also using a velcro arrangement may be attached/strapped near the elbow of the subject.

This design helps the subject take the measurement without needing to hold the sensor unit separately with another hand. The signal conditioning and data acquisition system may be then connected and made available in form a separate unit 208.

In one embodiment the sensors used include atleast one radial artery pressure sensor to receive radial artery pressure waveform and atleast one brachial artery pressure sensor to receive brachial artery pressure waveform. The sensor in each case might use an area-based sensor (a sensor with an area several times the width of the artery). The sensor used may include for eg. piezoelectric, polymer-based sensor

As shown in FIG. 2b a specialized sensor housing to firmly hold the pressure sensor 298 (eg. piezoelectric or polymer-based sensor) in contact with the skin with no moving parts may be provided. The housing may be symmetric and designed such that the cross section area of the housing reduces as a function of height from the bottom surface 296.

Further in one embodiment, the height of the housing may be less than the characteristic width of the sensor to ensure robustness to external vibrations. The cross-section area of the top of the housing in one embodiment may be at least one-third the area of the base of the housing (to ensure uniform distribution of pressure over the sensor area).

The symmetric design of the housing ensures a uniform distribution of pressure over the sensor area. This ensures application of only a modest pressure (much less than the arterial blood pressure) on the artery, thus preventing any occlusion or obstructed blood flow.

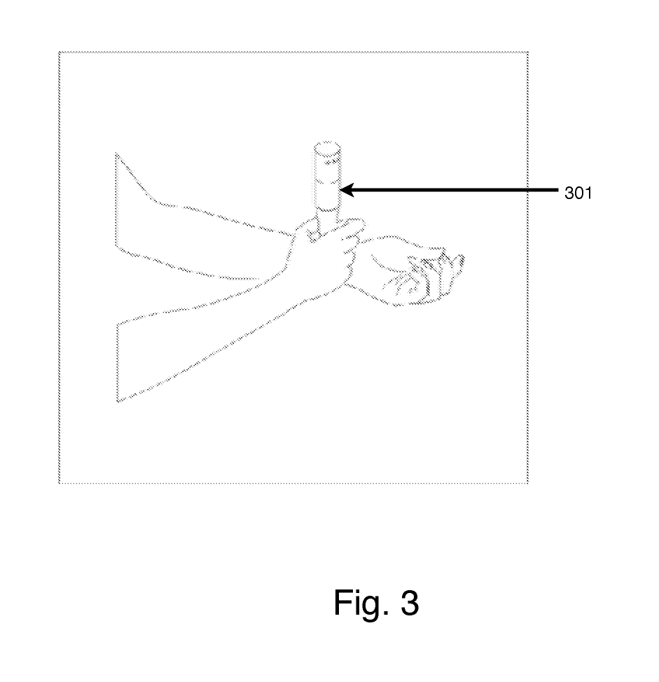

In another embodiment the signal conditioning and data acquisition system 104 may be integrated with the sensor system 102 in form of a pen 301 as shown in an embodiment of FIG. 3.

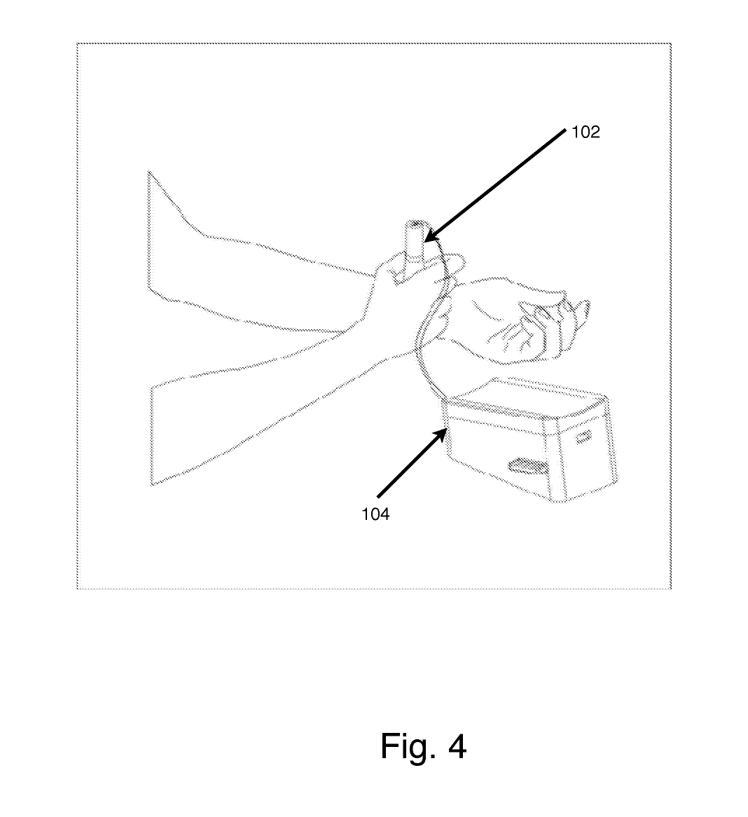

In another embodiment, the sensor system 102 and the signal conditioning and data acquisition system 104 is made available as a separate unit in a handheld design as shown in FIG. 4. In this embodiment the user or a practitioner may place the unit manually on the pulse point and hold it during the data collection.

In one embodiment herein, a switch may be provided in the sensor system to start the data recording. Initially, the waveforms may be analyzed visually/automatically to perfect the contact of the sensor system with the subjects' point of contact, for example subject's skin. The point of contact is not limited only to subject's limbs but can be extended to any point of interest on subject's body. After the point of contact is perfected, the system may be signaled to start recording the data for formal analysis. This could be done using a switch for starting and stopping data collection.

### C. Parameter Extraction and Signal Processing

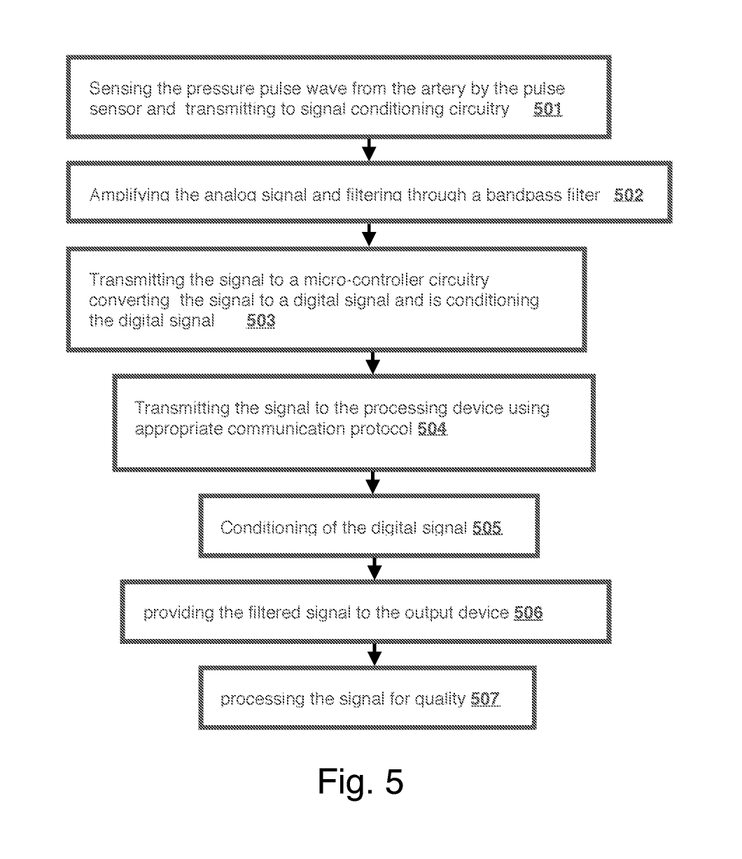

A method for health management using pulse detection, measurement and analysis as per an embodiment herein is shown in FIG. 5. The method comprises of sensing the pressure pulse wave from the artery by the pulse sensor and transmitting to signal conditioning circuitry 501. The analog signal is amplified and bandpass filtered 502 to reject high frequency components coming from environmental factors such as power line and very low frequency variations due to various kinds of motions. The method further comprises transmitting the signal to a microcontroller circuitry where it is converted to digital signal and is further conditioned 503. The method further comprises step of transmitting the digital signal to the processing device using appropriate communication protocol 504. In one embodiment the processing device can be microcontroller itself. Further, conditioning of the digital signal at the processing device takes place 505. In one embodiment the processing is done using phase preserving filtering and baseline wander correction.

Further step includes providing the filtered signal to the output device 506 (for display or other means of outputs). In one embodiment the filtered signal is further processed for its quality 507 using checks such as polarity reversal check, baseline shift check and signal to noise ratio check. In an embodiment the quality check also involves a subset of parameter extraction algorithm described below. It may further involve pattern recognition algorithm such as cross correlation and/or machine learning. This quality check helps in ensuring appropriate positioning of the sensor on the pulse point. A final output check is performed by the administrator (who could be same as subject) before initialing the final capture. Once data capture is initiated, the recorded data and or filtered data may be output in real-time.

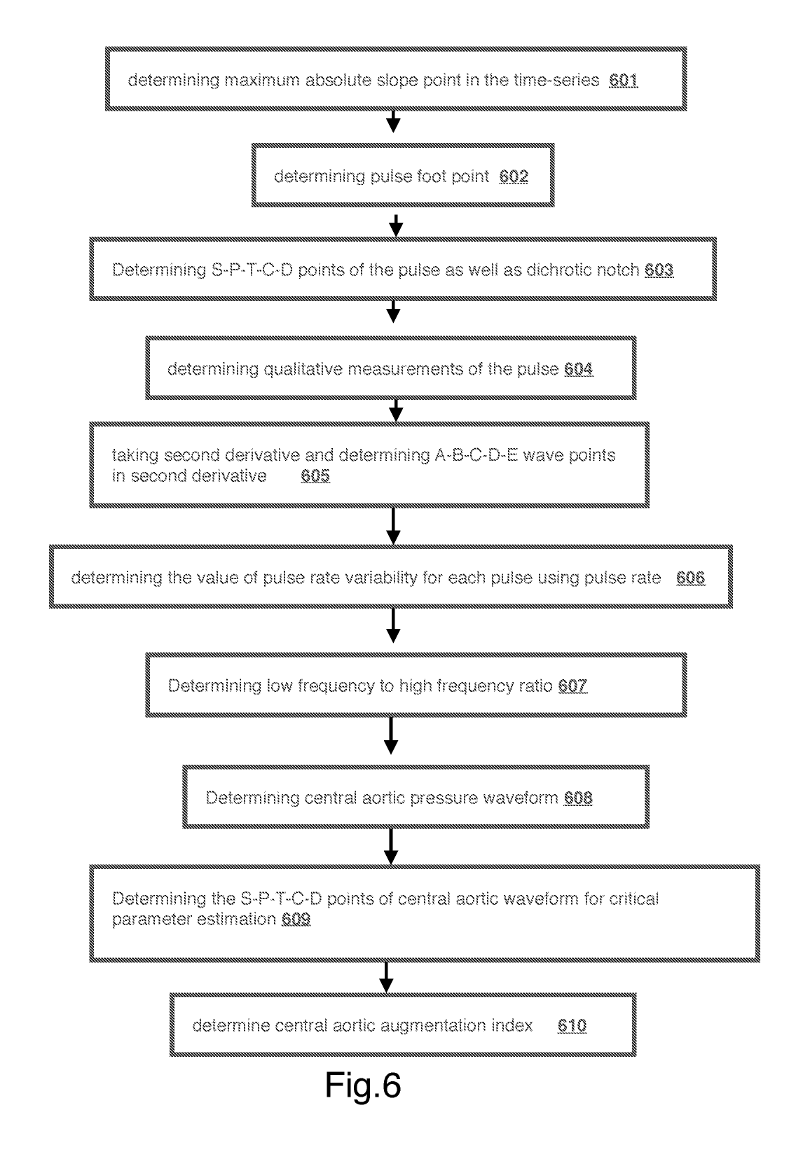

Steps of parameter extraction as per one embodiment herein is shown in FIG. 6. The parameter extraction of the recorded signal involves the steps of determining 601 maximum absolute slope point in the time-series. Further steps include determining 602 pulse foot point. Determination 603 S-P-T-C-D points of the pulse as well as dichrotic notch takes place.

Using the above determinations augmentation index, ejection duration, arterial stiffness index, Dichrotic Wave Amplitude, Pulse Height Variance, Pulse Width Variance, Relative Crest Time, Dichrotic Wave Time, Time Delay and others are determined 604. Further steps involve taking 605 second derivative and determining A-B-C-D-E wave points in second derivative. Using the pulse rate the value of pulse rate variability for each pulse is determined 606.

Determining 607 of low frequency to high frequency ratio by taking power spectral density of the pulse rate variability to determine critical points is performed. Waveform obtained from plurality of sensors is used to determine pulse wave velocity.

Central aortic pressure waveform is determined 608 using transfer function and various combinations of the parameters using pressure and/or volume waveforms from radial, brachial, carotid, femoral or any other arterial waveform, pulse wave velocity using one or more of these arterial waveform, and certain characteristics of the subject such as for example, Age, gender, height. Sometimes weight and/or ethinicity may also be used. The S-P-T-C-D points for central aortic waveform for critical parameter estimation are determined 609.

Critical parameters of central aortic waveform are used to determine 610 central aortic augmentation index. The central augmentation index helps in providing accurate evaluation of subject's cardiovascular health state and thus the indication of any imminent ailment. The system, apparatus and method for health management using pulse detection, measurement and analysis in various embodiments herein provides for an easy to use and increased accuracy measurement and analysis. This allows for implementation of the system in areas with shortage of medical practitioners. The various designs allow for variety of usage scenarios.

The foregoing description of the invention has been described for purposes of clarity and understanding. It is not intended to limit the invention to the precise form disclosed. Various modifications may be possible within the scope and equivalence of the claims.

1. A cardiovascular health monitoring device comprising:
atleast one radial artery pressure sensor to receive radial artery pressure waveform;
atleast one brachial artery pressure sensor to receive brachial artery pressure waveform;
a signal processing module configured to generate at least one of the cardiovascular parameter including peripheral augmentation index, pulse rate variability, arterial stiffness index, ejection duration, central aortic waveform, central aortic blood pressure and central augmentation index for analysis of cardiovascular health, based upon the received brachial artery pressure waveform and the received radial artery pressure waveform.

2. The cardiovascular health monitoring device as in claim 1, wherein the signal processing module is further configured to generate the ratio of low frequency to high frequency components in the fourier transform of the pulse rate variability.

3. The cardiovascular health monitoring device as in claim 1, wherein the signal processing module is further configured to generate pulse wave velocity.

4. The cardiovascular health monitoring device as in claim 3, wherein the signal processing module is further configured to receive physiological parameters of a subject and generate central aortic pressure waveform using the received brachial artery pressure waveform, the received radial artery pressure waveform, the pulse wave velocity and the received physiological parameters of the subject.

5. The cardiovascular health monitoring device as in claim 4, wherein the physiological parameters of the subject includes, age, gender, height and weight.

6. The cardiovascular health monitoring device as in claim 1, wherein, the atleast one radial artery pressure sensor and the atleast one brachial artery pressure sensor each comprises of:
a bottom surface in contact with the skin such that the area of contact with the skin is atleast multiple times that of the width of artery being sensed;
a sensor housing having cross-sectional area that reduces with height, starting from the bottom surface, wherein the height of the sensor housing is less than the width of the bottom surface of the sensor.

7. A non-transitory computer readable medium, having stored thereon, instructions that when executed by a computing device, cause the computing device to perform operations comprising:
receiving a radial artery pressure waveform;
receiving a brachial artery pressure waveform;
generating at least one of the cardiovascular parameter including peripheral augmentation index, pulse rate variability, arterial stiffness index, ejection duration, central aortic waveform, central aortic blood pressure and central augmentation index for analysis of cardiovascular health, based upon the received brachial artery pressure waveform and the received radial artery pressure waveform.

8. The non-transitory computer-readable medium as in claim 7, further having instructions stored thereon that, upon execution by one or more processors of a cardiovascular health monitoring device, cause the cardiovascular health monitoring device to carry out operations comprising:
generating the ratio of low frequency to high frequency components in the fourier transform of the pulse rate variability.

9. The non-transitory computer-readable medium as in claim 7, further having instructions stored thereon that, upon execution by one or more processors of a cardiovascular health monitoring device, cause the cardiovascular health monitoring device to carry out operations comprising:
generating pulse wave velocity.

10. The non-transitory computer-readable medium as in claim 7, further having instructions stored thereon that, upon execution by one or more processors of a cardiovascular health monitoring device, cause the cardiovascular health monitoring device to carry out operations comprising:
receiving physiological parameters of a subject; and
generating central aortic pressure waveform using the received brachial artery pressure waveform, the received radial artery pressure waveform, the pulse wave velocity and the received physiological parameters of the subject.

11. A non-transitory computer-readable medium as in claim 10 wherein the physiological parameters of the subject includes, age, gender, height and weight.
